(19) United States
(12) Patent Application Publication
(10) Pub. No.: US 2007/0191874 A1
(43) Pub. Date: Aug. 16, 2007

(54) Devices and Methods for Performing Surgical Procedures

(75) Inventor: Benjamin C. Sun, Columbus, OH (US)

Correspondence Address: CALFEE HALTER & GRISWOLD, LLP, 800 SUPERIOR AVENUE, SUITE 1400, CLEVELAND, OH, 44114, US

(73) Assignee: The Ohio State University Research Foundation, Columbus, OH (US)

(21) Appl. No.: 11/559,869

(22) Filed: Nov. 14, 2006

Related U.S. Application Data
(60) Provisional application No. 60/736,347, filed on Nov. 14, 2005.

(51) Int. Cl. A61B 17/32

(52) U.S. Cl. 606/184

(57) ABSTRACT

Devices and methods allow surgical procedures to be performed on an organ without significant loss of fluid, such as, for example, surgical procedures on a beating heart. The devices include an expandable obstruction for insertion into the organ to block an opening in the tissue, and an access port with one or more conduits for accessing the interior of the organ.

### CROSS REFERENCE TO RELATED APPLICATIONS

This application claims priority to and any other benefit of U.S. Provisional Application Ser. No. 60/736,347, filed on Nov. 14, 2005, the entire content of which is incorporated by reference herein.

### FIELD OF THE INVENTION

The present disclosure relates to devices and methods which provide access to the interior of chambered tissues and organs, such as the heart, so that a variety of surgical procedures may be performed. The devices and methods may be used, for example, for performing surgical procedures on the heart while the heart is beating.

### BACKGROUND

There are a variety of surgical procedures which require access to fluid filled organs in an animal, such as, for example, the heart or a portion of the heart. In one example, the left ventricle of the heart is accessed in order to perform surgery, such as for the implementation of a left ventricular assist device (LVAD). Often, when one or more chambers of the heart is accessed, the patient is placed on a heart-lung machine and then the heartbeat of the patient is suspended. The heart lung machine artificially delivers oxygen to the bloodstream, maintains body temperature, and pumps blood through the body during the surgical procedure. There are many disadvantages to the use of heart-lung machines. In order to avoid clotting, the patient's blood must be thinned, requiring the administration of heparin or another blood-thinning agent. Furthermore, components of the patient's blood may become damaged upon repeated circulation through the heart-lung machine. As a result, many patients require transfusions in the post-operative period. In addition, blood circulation through the machine may trigger an inflammatory response, resulting in fever and sequestration of fluids in the lungs. There is also an increased risk of stroke.

For these reasons, it is desirable to perform surgical procedures in the heart without requiring that the heart be stopped during surgery.

### SUMMARY

The present application relates to devices and methods which allow surgical procedures to be performed on an organ without significant loss of fluid, such as, for example, surgical procedures on a beating heart. According to the various embodiments of the invention, a device is provided that includes an expandable obstruction for insertion into the organ to block an opening in the tissue, such as vasculature, or branches within the heart, or an incision or other opening within the heart tissue, so that fluid flow through the opening is occluded In one embodiment, the expandable obstruction comprises at least one inflatable balloon component, which may be inserted through the opening in a deflated state, and then inflated to effectively block the opening.

According to the various embodiments, the device includes an access port, such as, for example, a hollow shaft, which may be inserted into an incision in the organ to deliver one or more surgical tools into the organ, such as, for example, an expandable obstruction for blocking the incision, a light source or camera for viewing the internal tissue, or a tool for the extraction of thrombus from within the organ. In one such embodiment, a relatively rigid access port configured for inserted into an incision of the organ may be used to insert a deflated balloon component into the organ. In various embodiments, one or more conduits may be connected with the balloon component and extend through the access port to connect to a source or pressurized gas may be used to inflate the inserted balloon component, thereby occluding the flow of fluid through the incision. In yet other embodiments, one or more additional conduits may extend through the access port for delivery of tools, or for provision of irrigation or drainage.

According to some embodiments, the device may be configured to facilitate illumination of an internal cavity of the organ. According to some such embodiments, an expandable obstruction may include a reflective inner layer and a light transmitting outer layer, with a light source disposed between the inner layer and the outer layer, such that light emitted from the light source may reflect off of the inner layer and be directed through the outer layer to illuminate the internal cavity in which the obstruction has been implanted. In one such embodiment, the inner and outer layers may include inner and outer balloon components, which may, for example, be inserted through an incision in the organ while in a deflated condition, and then inflated to provide an expanded light source. In other embodiments, visualization or illumination are achieved using other sources, which may be inserted into the organ through one or more additional conduits.

According to yet other embodiments, a method is provided for preparing an organ for a surgical procedure. In various embodiments of the method, an incision is formed in the organ, and an access port is inserted through the incision. A balloon component is delivered through the access port and into the incision, and the balloon component is then inflated. In some embodiments, a core of tissue around the incision may be removed, for example, through the use of a coring knife surrounding the access port, to produce an opening in the organ that is obstructed by the balloon component. A surgical procedure may then be performed on the organ.

### BRIEF DESCRIPTION OF THE DRAWINGS

FIG. 1 is a side schematic view of a surgical device;

FIGS. 2A and 2B are side and end views of a coring knife portion of a surgical device;

FIG. 3 is a side schematic view, shown in partial cross-section, of a balloon component of a surgical device;

FIG. 4 is a side schematic view or a access port portion of a surgical device; and

FIG. 5 is a side view of a coring knife portion of a surgical device.

### DETAILED DESCRIPTION

While the invention has been described with reference to specific embodiments, it will be apparent to those skilled in the art that may alternatives, modifications, and variations may be made. Accordingly, the present invention is intended to embrace all such alternatives, modifications, and variations that may fall within the spirit and scope of the appended claims, which follow. Further, while the various embodiments described herein relate specifically to surgical procedures involving the heart, the present application contemplates use of the inventive devices and methods for surgical procedures involving any fluid containing organ, as will be understood in the art.

To safely maintain the circulation of blood during a surgical procedure performed on the heart, for example, where a significant opening or hole is created in the heart, an expandable obstruction or occluder may be inserted into the heart to occlude blood flow out of the opening and allow the heart to continue to circulate blood through the body.

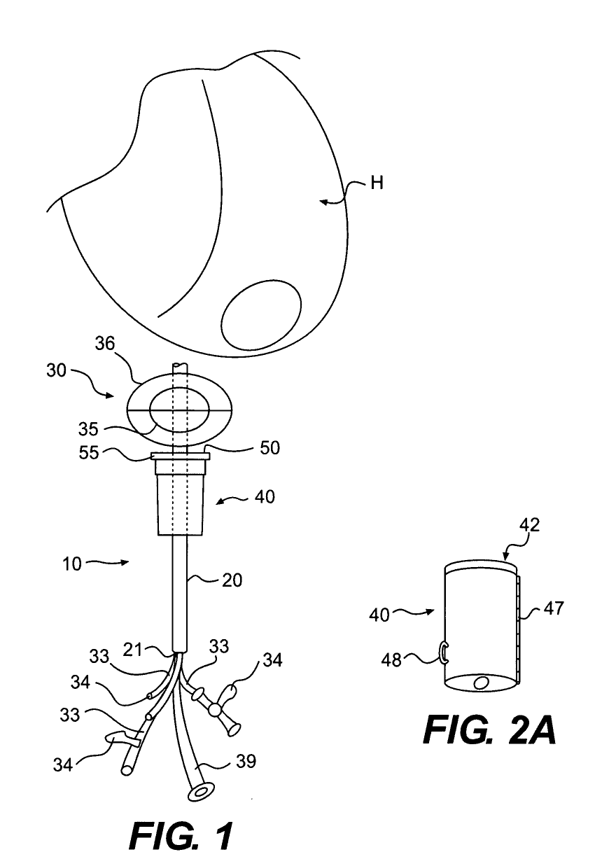

FIG. 1 illustrates a surgical device 10 configured to provide access to the heart H, such as, for example, thorough an opening produced in the wall of the heart, to assist in the performance of surgical procedures which may, but need not, be performed while the heart is still beating. The surgical device includes an access port 20, which may be configured to be inserted into an incision in the heart. As shown, the access port 20 may include a narrow, hollow shaft 21, sized to reduce the size of the incision required in the heart H. The shaft 21 may, but need not, be relatively rigid to provide stability and support for one or more tools inserted through the shaft 21 and into the incision.

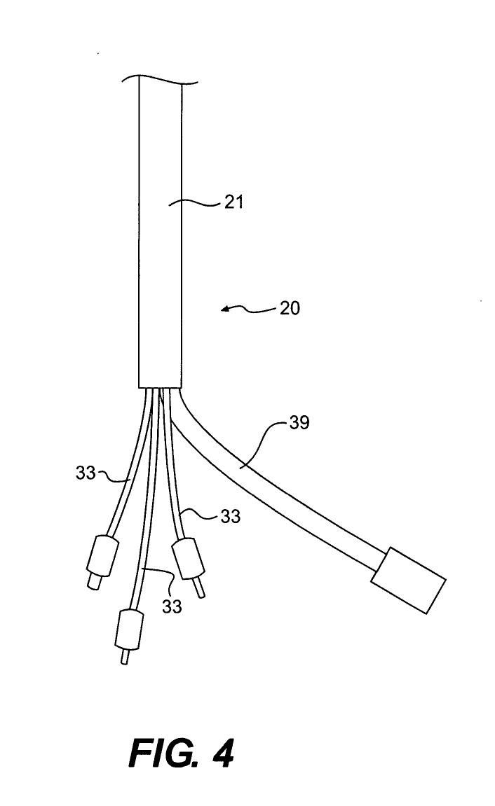

Many different types of tools or surgical components may be inserted into the heart H through the access port 20, including, for example, cameras or light sources (including fiber optics), extraction tools, catheters, or an obstruction or occluder. In one embodiment as illustrated in FIG. 1, the device 10 may include an expandable obstruction or occluder 30, that is insertable through the access port 20 and into the heart incision while in a deflated or partially deflated condition. As shown in FIG. 4, one or more conduits 33 may be connected with the obstruction 30, such that pressurized fluid may be supplied through the conduits 33 to the obstruction 30 to inflate the obstruction for effective blockage of an opening produced around the incision. In some embodiments, the conduits 33 may be provided with valves, such as stopcocks 34, to regulate the supply of pressurized fluid to the obstruction 30. While the illustrated embodiment shows the expandable obstruction 30 insertable into a heart chamber through an opening at the distal end of the hollow shaft 21, the obstruction 30 may alternatively or additionally extend through an opening in the side or the hollow shaft 21 (not shown) Further, an additional opening or openings may be provided in the shaft 21 to facilitate the performance of additional surgical procedures, such as, for example, suction or irrigation, through the use of additional tools or conduits receivable within the shaft 21.

In other embodiments, an expandable obstruction or occluder may be configured to facilitate illumination of an internal cavity of the heart. In one embodiment, an obstruction may include an inner layer with a reflective external surface, an outer light transmitting layer, and a light source disposed between the inner and outer layers. In such an embodiment, light emitted from the light source is reflected off of the inner layer and directed outward through the light transmitting layer to illuminate the cavity in which the obstruction has been inserted. In the embodiment of FIGS. 1 and 3, the obstruction 30 includes an inner balloon member 35 having a reflective outer surface, an outer balloon member 36, at least a portion of which is light transmitting, and a fiber optic light source 38, as shown in FIG. 3, disposed between the inner balloon member 35 and the outer balloon member 36. After the obstruction is inserted through the access port 20 and into the incision in the heart H, the inner and outer balloon members 35, 36 may be inflated by supplying pressurized fluid through conduits 33. Power to the light source 38 may then be supplied through a cable 39 disposed in the access port 20, to illuminate the light source 38. Light emitted from the light source 38 is reflected off of the inner balloon member 35 and emitted through the light transmitting portions of the outer balloon member 36. In other embodiments (not shown), an additional outer balloon member may surround outer balloon member 36, which may facilitate control, positioning, and sizing of the obstruction, or provide for spacing of the light source or other internal tools from the occluded tissue. A scope, fiber optic camera or other such means of inspection may be inserted through the access port 20 to visually inspect the cavity of the heart H in which the obstruction 30 has been implanted. Where a thrombus, clot, or other such matter is discovered, the obstruction 30 may be deflated and removed to allow for insertion of an extraction tool to remove the unwanted matter.

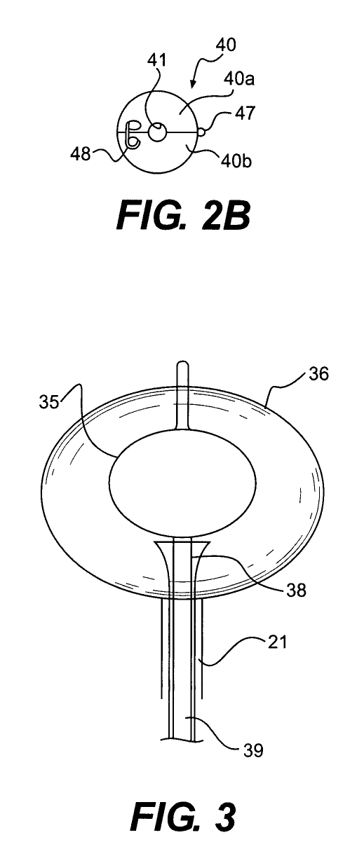

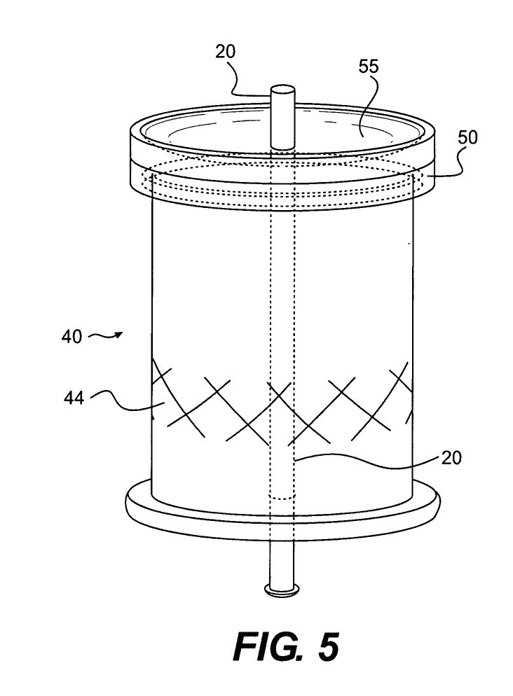

Some surgical procedures may require a larger hole or opening in the heart, such as for example, implantation of a ventricle assist device (VAD), insertion of a cannula to the wall of the heart. To produce such an opening in the heart H, the device 10 may further be provided with a cutting mechanism to cut an opening in the heart proximate to the incision in which the access port 20 has been inserted. In one embodiment, the cutting mechanism includes a cylindrical coring knife that is slidable along the access port to engage the heart tissue surrounding the incision, such that the engaged coring knife may cut into the heart tissue to remove a core of heart tissue and produce an opening in the heart wall. While many different types of coring knives, as known in the art, may be used, in the illustrated example shown in FIGS. 2A and 2B, a cylindrical coring knife 40 includes an aperture 41 along a central axis through which the access port 20 may be received. The coring knife 40 slides along the access port 20 to engage a sharpened end 42 or blade of the knife 40 with the wall of the heart H surrounding the incision. The illustrated coring knife 40 may be manually rotated and pressed against the heart wall to cut a core of material and produce an opening in the heart. Referring to FIG. 5, a textured outer surface 44 may be provided to facilitate grasping the coring knife 40 during operation of the knife.

While a coring knife may be slid off of the proximal end of the access port 20 to remove the coring knife from the device, for example, to access the heart with a different surgical tool in the illustrated embodiment, a proximal end of the access port may include a flange 25 to prevent the coring knife 40 from sliding off of, and inadvertently separating from, the access port 20. According to the embodiment shown in FIG. 2A, to allow for removal of the illustrated coring knife 40 from the access port 20, the coring knife 40 may include two sections 40a, 40b connected by a hinge 47 and a clasp 48, as shown in FIGS. 2A and 2B. When the clasp 48 is disengaged, the coring knife sections 40a, 40b may pivot apart on hinge 47, such that the coring knife 40 may be removed from the access port 20.

Referring to FIGS. 1 and 5, the device may further be provided with a plate member 50 positionable on the access port 20, and may be sized to be received within the hollow blade of the coring knife 40. The plate member 50 includes a distal surface 55 that is facing the heart when the device is in use, and is configured to engage and stabilize the outer surface of the heart, and to retain the core of heart tissue removed by the coring knife 40. The distal surface 55 may also be concave or otherwise shaped to support and protect the inflated obstruction 30 during the cutting operation. In some embodiments, the distal surface 55 of the plate 50 may be textured to improve contact with the surface of the organ. The surface 55 may also have a diameter large enough to fully retain the removed core of heart tissue. In some embodiments the plate is adapted to be removed from the access port 20, such as by use of hinges and clasps, or other means.

The components of the illustrated device 10 may be provided in many different materials, including, for example, surgical grade or bio-compatible metals, plastics, and ceramics, as known in the art. It will be understood that different components of the illustrated device 10 may be made of different materials or the same materials.

In one method of utilizing the above described embodiment of a device according to the invention, a small incision is made in the heart, for example, in the apex of the left ventricle. A device as illustrated in FIG. 1 is used. A distal end of the access port 20, which contains the obstruction 30 is inserted into the incision. The obstruction 30 is inflated by supplying pressurized fluid through conduits 33, first to the outer balloon member 36 and then to the inner balloon member 35. Power is supplied to the light source 38 through cable 39 to illuminate the internal cavity of the heart, for example, the left ventricle. This illumination is facilitated by reflection of emitted light off of the reflective external surface of the inner balloon member 35 to pass through the light transmitting portions of the outer balloon member 36. The heart may then be inspected, for example, using a fiber optic camera (not shown) inserted into the heart through the access port 20. If a clot or thrombus or other such problem is discovered, the obstruction 30 may be deflated and removed for appropriate surgical treatment.

Once the obstruction 30 has been inflated, a larger hole is produced in the wall of the heart around the incision, for example, by using a coring knife 40 slidable along the access port 20 to engage the heart wall and rotatable to remove a core of tissue from the heart wall. The inflated obstruction 30 may be positioned to block the hole, thereby preventing significant blood loss through the hole, allowing the heart H to continue to circulate blood. The removed core of heart tissue may be received in the plate 50 disposed between the coring knife 40 and the heart H. The clasp 48 of the coring knife 40 may be disengaged, allowing the coring knife sections 40a, 40b to pivot open about hinge 47, and the coring knife 40 may be removed from the access port. The plate 50 is then allowed to slide off of the access port 20 to remove the core of heart tissue from the body. The hole produced in the heart may then be accessed for a surgical procedure, such as, for example, attachment of a cannula to the hole for implantation of a ventricle assist device.

In some embodiments, assembly or disassembly of the device from its individual components may be necessary. The access port 20 may be reusable and may be manufactured separately, with the obstruction 30 and inflation tubing 33 introduced into the device 10 at a later time. The balloon members 35, 36 may be disposable, and thus new balloon members 35, 36 may be introduced into the device 10 each time it is used. Additionally, while specific coring knives 40 are described herein, it is possible to use the device with a coring knife already existing in the art.

Further advantages and benefits of the invention will become apparent to those skilled in the art after considering the accompanying drawings.

While various inventive aspects, concepts and features of the inventions may be described and illustrated herein as embodied in combination in the various embodiments these various aspects, concepts and features may be used in many alternative embodiments, either individually or in various combinations and sub-combinations thereof. Unless expressly excluded herein all such combinations and sub-combinations are intended to be within the scope of the present inventions. Still further, while various alternative embodiments as to the various aspects, concepts and features of the inventions—such as alternative materials, structures, configurations, methods, circuits, devices and components, software, hardware, control logic, alternatives as to form, fit and function, and so on—may be described herein, such descriptions are not intended to be a complete or exhaustive list of available alternative embodiments, whether presently known or later developed. Those skilled in the art may readily adopt one or more of the inventive aspects, concepts or features into additional embodiments and uses within the scope of the present inventions even if such embodiments are not expressly disclosed herein. Additionally, even though some features, concepts or aspects of the inventions may be described herein as being a preferred arrangement or method, such description is not intended to suggest that such feature is required or necessary unless expressly so stated. Still further, representative values and ranges may be included to assist in understanding the present disclosure; however, such values and ranges are not to be construed in a limiting sense and are intended to be critical values or ranges only if so expressly stated. Moreover, while various aspects, features and concepts may be expressly identified herein as being inventive or forming part of an invention, such identification is not intended to be exclusive but rather there may be inventive aspects, concepts and features that are fully described herein without being expressly identified as such or as part of a specific invention, the inventions instead being set forth in the appended claims. Descriptions of methods or processes are not limited to inclusion of all steps as being required in all cases, nor is the order that the steps are presented to be construed as required or necessary unless expressly so stated.

1. A device for preparing a heart for a surgical procedure, the device comprising:
an access port comprising a hollow shaft having a distal end for insertion into the heart through an incision in the heart and a proximal end providing access to the hollow shaft after insertion into the heart;
an obstruction, configured to be inserted into the heart through the access port; and
a coring knife, assembled with the access port and configured to remove a core of heart tissue surrounding the access port to produce an opening in the heart,
wherein the inserted obstruction is configured to occlude blood flow through the heart opening.

2. The device of claim 1, wherein the obstruction comprises a balloon component that is expandable.

3. The device of claim 2, wherein the balloon component is expandable by inflation.

4. The device of claim 3, wherein the inflatable component is inflatable by supplying pressurized fluid through one or more conduits connected with the inflatable component, the one or more conduits extending from the proximal end of the hollow shaft.

5. The device of claim 1, wherein the coring knife comprises a cylindrical blade portion and an aperture along a central axis allowing the coring knife to slide along the access port.

6. The device of claim 5, wherein the coring knife comprises first and second coring knife sections joined by a hinge portion and a clasp portion to define the aperture, such that disengagement of the clasp portion allows the first and second coring knife sections to pivot about the hinge portion for removal of the coring knife from the access port.

7. The device of claim 1, further comprising a plate portion assembled with the access port distal to the coring knife.

8. The device of claim 7, wherein the plate portion is configured to receive the removed core of heart tissue.

9. A device for preparing a heart for a surgical procedure, the device comprising:
an access port comprising a hollow shaft having a distal end for insertion into the heart through an incision in the heart and a proximal end providing access to the hollow shaft after insertion into the heart; and
an expandable obstruction, configured to be inserted into the heart through the access port and inflatable by supplying pressurized fluid through one or more conduits connected with the expandable obstruction, the one or more conduits extending from the proximal end of the hollow shaft,
wherein the inserted and inflated expandable obstruction is configured to occlude blood flow through an opening in the heart.

10. The device of claim 9, wherein the inflatable component comprises an inner balloon member, an outer balloon member surrounding the inner balloon member, and a light source disposed between the inner balloon member and the outer balloon member.

11. The device of claim 10, wherein the inner balloon member comprises a reflective external surface, and the outer balloon member comprises a light transmitting portion.

12. The device of claim 9, further comprising a coring knife, assembled with the access port and configured to remove a core of heart tissue surrounding the access port to produce an opening in the heart.

13. The device of claim 12, further comprising a plate portion assembled with the access port distal to the coring knife.

14. The device of claim 13, wherein the plate portion comprises a distal surface shaped to protect the inflated expandable obstruction.

15. The device of claim 14, wherein at least a portion of the distal surface is concave to conform to an outer surface of the inflated expandable obstruction.

16. The device of claim 13, wherein the plate portion is configured to receive the removed core of heart tissue.

17. A method for preparing a heart for a surgical procedure, comprising:
creating a small incision in the heart;
inserting an expandable obstruction into the heart through the incision;
inflating the expandable obstruction; and
removing a core of heart tissue surrounding the incision to produce an opening in the heart,
wherein the inflated obstruction occludes blood flow through the opening, such that a surgical procedure may be performed on the heart while the heart is still beating.

18. The method of claim 17, wherein inserting an expandable obstruction into the heart through the incision comprises inserting the expandable obstruction into a hollow shaft and inserting a distal end of the hollow shaft into the incision.

19. The method of claim 17, wherein removing a core of heart tissue surrounding the incision to produce an opening in the heart comprises inserting a shaft into the incision and engaging a coring knife with the heart, wherein the coring knife is slidable on the shaft.

20. A device for preparing the organ for a surgical procedure, the device comprising:
an access port comprising a hollow shaft having a distal end for insertion into the organ through an incision in the organ and a proximal end providing access to the hollow shaft after insertion into the organ;
an obstruction, configured to be inserted into the organ through the access port; and
a coring knife, assembled with the access port and configured to remove a core of tissue surrounding the access port to produce an opening in the organ,
wherein the inserted obstruction is configured to occlude fluid flow through the opening.

21. A method for preparing the organ for a surgical procedure, comprising:
creating a small incision in the organ;
inserting an expandable obstruction into the organ through the incision;
inflating the expandable obstruction; and
removing a core of tissue surrounding the incision to produce an opening in the organ,
wherein the inflated obstruction occludes fluid flow through the opening, such that a surgical procedure may be performed on the organ without significant loss of fluid.
